(19) United States
(12) Patent Application Publication
(10) Pub. No.: US 2026/0086063 A1
(43) Pub. Date: Mar. 26, 2026

(54) METHODS OF DIAGNOSING ACUTE CIRCULATORY FAILURE

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); CENTRE HOSPITALIER UNIVERSITAIRE DE TOULOUSE, Toulouse (FR); UNIVERSITÉ TOULOUSE III - PAUL SABATIER, Toulouse (FR)

(72) Inventors: Anne GALINIER, Toulouse (FR); Cédric DRAY, Toulouse (FR); François LABASTE, Toulouse (FR); Marion STEPHAN, Toulouse (FR); Vincent PEY, Toulouse Cedex (FR); Vincent MINVILLE, Toulouse (FR)

(21) Appl. No.: 19/110,270

(22) PCT Filed: Oct. 5, 2023
(86) PCT No.: PCT/EP2023/077580

§ 371 Date: Mar. 10, 2025

(30) Foreign Application Priority Data
Oct. 6, 2022 (EP) 22306499.9

(51) Int. Cl. G01N 27/327; G01N 33/49

(52) U.S. Cl. CPC G01N 27/3277; G01N 33/49; G01N 2800/325; G01N 2800/52

(57) ABSTRACT

Circulatory failure generates hypoxia and leads to accumulation of reductive species in tissue and circulatory failure monitoring tools are needed but rare. Cardiopulmonary bypass (CPB) is known to promote brief circulatory failure during its initiation. In the present study, the Inventors demonstrate a correlation between whole blood redox potential and circulatory failure during (CPB). They made a prospective study with 17 patients eligible for cardiac surgery with cardiopulmonary bypass. They demonstrated a frank reduction of the whole blood redox potential during circulatory failure during the initiation of CPB. They also demonstrated that they were able to classify patients in 3 groups, one of them presenting an unfavorable post-operative outcome. Accordingly, the present invention relates to a method of diagnosing acute circulatory failure in a patient comprising determining the level of redox potential in a sample obtained from said patient, wherein the level of redox potential indicates whether the patient suffers or not from an acute circulatory failure.

### FIELD OF THE INVENTION

The present invention is in the field of medicine, in particular vascular disorders.

### BACKGROUND OF THE INVENTION

Circulatory failure is an alteration of tissue perfusion associated with cellular response to a drop in oxygenation (1). Circulatory failure is characterized by impaired oxygen transport, leading to tissue dysoxia (2). The main clinical consequences are hypotension and organ failure. Several diseases can lead to the occurrence of circulatory failure. When a patient suffers from circulatory failure, the duration of hospitalization is then increased as well as morbidity and mortality (3,4). Prompt and appropriate care is therefore recommended with the aim of reducing morbidity and mortality (5,6). In order to monitor the evolution and quality of care, some biomarkers have demonstrated their usefulness (7). Lactatemia is particularly useful for the diagnosis and evaluation of the prognosis during circulatory failure. However, the measurement of lactate meet some limitations because of non-hypoxic causes of elevated lactate such as hepatic dysfunction, pyruvate dehydrogenase dysfunction and accelerated glycolysis (8). A lactate/pyruvate ratio in blood samples is more specific than blood lactate alone to identify tissue hypoxia. However, the dosage of blood pyruvate requires a specific technical platform and cannot be carried out in current practice for the regular monitoring of patients (9). Accordingly, the identification of new markers for screening and monitoring circulatory failure is still mandatory.

### SUMMARY OF THE INVENTION

The present invention is defined by the claims. In particular, the present invention relates to a method of diagnosing acute circulatory failure in a patient comprising determining the level of redox potential in a sample obtained from said patient, wherein the level of redox potential indicates whether the patient suffers or not from an acute circulatory failure.

### DETAILED DESCRIPTION OF THE INVENTION

Here, the Inventors describe the usefulness of monitoring the evolution of whole blood Eg in a patient suffering from acute circulatory failure. In this work, they chose to study a state of programmed acute circulatory failure, namely extracorporeal circulation during cardiac surgery.

### Diagnostic Methods

In a first aspect, the present invention relates to a method of diagnosing acute circulatory failure in a patient comprising determining the level of redox potential in a sample obtained from said patient, wherein the level of redox potential indicates whether the patient suffers or not from an acute circulatory failure.

As used herein, the term “Acute Circulatory Failure” or “ACF” refers to a clinical syndrome characterized by inadequate effective blood flow and reduced tissue perfusion with decreased delivery of oxygen. More particularly, ACF results from an imbalance of systemic venous return and cardiac function, leading to a decrease in cardiac output and to systemic organ insufficiency. The reduction in oxygen delivery leads to impaired oxidative metabolism, lactic acidosis and cell death. Acute circulatory failure may be due to primary myocardial disease such as myocarditis, cardiomyopathy, cardiac allograft failure or congenital heart disease, or secondary to systemic conditions such as sepsis and other inflammatory processes. The diagnosis of acute circulatory failure is established by noninvasive and invasive methods. Noninvasive methods include as example near-infrared spectroscopy (NIRS) and measurement of lactate level and/or pyruvate level. Invasive methods include as example central venous pressure monitoring, co-oximetry and assessment of cardiac output via transpulmonary thermodilution. In some embodiments, the acute circulatory failure is not due to sepsis. In some embodiments, the acute circulatory failure is initiated by a medical procedure. In some embodiments, the acute circulatory failure is initiated by extracorporeal circulation. In some embodiments, the acute circulatory failure is initiated by extracorporeal circulation during cardiac surgery. In some embodiments, the acute circulatory failure is induced by cardiopulmonary by-pass.

As used herein, the term “patient” denotes a mammal. Typically, a subject according to the invention refers to any subject (preferably human) afflicted with or susceptible to be afflicted with an acute circulatory failure. In a preferred embodiment, the patient is human. In some embodiments, the patient is a man. In some embodiments, the patient is a woman. In some embodiments, the subject is an infant, a child, a teen, an adult or a senior. In some embodiments, the subject is at least 60 years old. In some embodiments, the subject is at least 72 years old.

As used herein, the term “redox potential” or “E_{g}” refers to a measure of the tendency of chemical species to acquire electrons from or lose electrons to an electrode and thereby be reduced or oxidized respectively. Typically, the measurement of the redox potential is carried out using an electrode. The measuring electrode is immediately immersed in a sample of the patient's arterial whole blood without pre-treatment. Thus, in some embodiments, the level of redox potential is measured with an electrode immersed in said sample without pre-treatment. In some embodiments, the electrode is a platinum electrode. Typically, the data is collected for 75 seconds using an informatic interface (as example an application such as NOVA® 2.0). The redox potential value is obtained after stabilization of the potential after about 30 seconds. Those skilled in the art well known other methods to measure redox potential.

As used herein, the term “sample” refers to a biological sample obtained for the purpose of in vitro evaluation. Typical biological samples to be used in the method according to the invention are whole blood samples. In some embodiments, said biological sample is a biological liquid sample selected in the group consisting of whole blood, plasma, serum, saliva, urea and exudates. Thus, in some embodiments, the sample is chosen from whole blood sample, plasma sample, serum sample, saliva sample, urea sample and exudate sample. Preferably, the sample is a whole blood sample. In some embodiments, the sample is an arterial whole blood sample. In some embodiments, when the acute circulatory failure is initiated by extracorporeal circulation or cardiopulmonary by-pass, the sample obtained from said patient is collected during the initiation of said extracorporeal circulation or said cardiopulmonary by-pass.

In some embodiments, the present invention relates to a method of diagnosing acute circulatory failure in a patient comprising determining the level of redox potential in a sample obtained from said patient, wherein a downregulation of said redox potential as compared to a predetermined reference value indicates that the patient suffers from acute circulatory failure.

In some embodiments, the present invention relates to a method of diagnosing acute circulatory failure in a patient comprising determining the level of redox potential in a sample obtained from said patient, wherein an upregulation of said redox potential as compared to a predetermined reference value indicates that the patient suffers from acute circulatory failure.

In some embodiments, the present invention relates to a method of diagnosing acute circulatory failure in a patient comprising determining the level of redox potential in a whole blood sample obtained from said patient, wherein a downregulation of said redox potential as compared to a predetermined reference value indicates that the patient suffers from acute circulatory failure.

Typically, said redox potential is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% down- or upregulated as compared to said predetermined reference value. In some embodiments, the redox potential is at least 20% down- or upregulated as compared to said predetermined reference value.

In some embodiments, the predetermined reference value is relative to a number or value derived from population studies, including without limitation, subjects of the same or similar age range, subjects in the same or similar ethnic group, and subjects having the same severity of lesion. Such predetermined reference values can be derived from statistical analyses and/or risk prediction data of populations obtained from mathematical algorithms and computed indices. In some embodiments, retrospective measurement of the level of the marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWERSAS, CREATE-ROC.SAS, GB STAT VI0.0 (Dynamic Microsystems, Inc. Silver Spring, Md., USA), etc. In some embodiments, the predetermined reference value is a redox potential value obtained from the patient before acute circulatory failure.

### Prognostic Methods

In a second aspect, the present invention relates to a method of predicting the outcome of a patient after an acute circulatory failure comprising determining the level of redox potential in a sample obtained from said patient, wherein the level of redox potential indicates the outcome.

As used herein, the term “after acute circulatory failure” refers to an event occurring later in time than the initiation of acute circulatory failure. In some embodiments, the level of redox potential is determined in a sample collected at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 minutes after the initiation of the acute circulatory failure. In some embodiments, the level of redox potential is determined in a sample collected at least 10 minutes after the initiation of the acute circulatory failure. In some embodiments, the acute circulatory failure is initiated by a medical procedure. In some embodiments, the acute circulatory failure is initiated by extracorporeal circulation. In some embodiments, the acute circulatory failure is initiated by extracorporeal circulation during cardiac surgery.

In some embodiments, the present invention relates to a method of predicting the outcome of a patient after an acute circulatory failure comprising determining the level of redox potential in a sample obtained from said patient, wherein an upregulation of said redox potential as compared to a predetermined reference value indicates that the patient has a poor outcome.

In some embodiments, the present invention relates to a method of predicting the outcome of a patient after an acute circulatory failure comprising determining the level of redox potential in a sample obtained from said patient, wherein a downregulation of said redox potential as compared to a predetermined reference value indicates that the patient has a poor outcome.

In some embodiments, the sample is a whole blood sample. In some embodiments, the sample is an arterial whole blood sample. In some embodiments, the present invention relates to a method of predicting the outcome of a patient after an acute circulatory failure comprising determining the level of redox potential in a whole blood sample obtained from said patient, wherein an upregulation of said redox potential as compared to a predetermined reference value indicates that the patient has a poor outcome.

In some embodiments, the present invention relates to a method of predicting the postoperative outcome of a patient after an acute circulatory comprising determining the level of redox potential in a sample obtained from said patient wherein the level of redox potential as compared to a predetermined reference value indicates the postoperative outcome.

In some embodiments, the present invention relates to a method of predicting the postoperative outcome of a patient after an acute circulatory failure induced by cardiopulmonary by-pass (CPB) comprising determining the level of redox potential in a sample obtained from said patient wherein the level of redox potential as compared to a predetermined reference value indicates the postoperative outcome.

As used herein the term “surgery” refers to any manual or operative methods or manipulations for the treatment or prevention of disease, injury or deformity. Surgery includes methods or manipulations conducted while a subject is under anesthesia, including local or general anesthesia. Surgery can be performed by a doctor, surgeon or dentist, generally in a hospital or other health care facility. Subjects undergoing surgery can be hospitalized or ambulatory, e.g., out-subject surgery. For purposes of this invention surgery includes, but is not limited to: abdominal surgery (e.g. surgery of the abdominal viscera), bench surgery (e.g. surgery performed on an organ that has been removed from the body, after which it can be reimplanted), cardiac (e.g. surgery of the heart), cerebral (e.g. surgery upon the brain), cineplastic (e.g. surgery to create a tunnel through a muscle adjacent to the stump of an amputated limb, to permit use of the muscle in operating a prosthesis), cosmetic (e.g. surgery to improve a subject's appearance by plastic restoration, correction or removal of blemishes), dentofacial (e.g. surgery involving defects of the face and structures of the mouth), neurological (e.g. surgery involving the peripheral or central nervous system), oral (e.g. surgery involving defects of the mouth, jaws and associated structures), orthopedic (e.g. surgery dealing with bones and bony structures such as hip replacement), pelvic (e.g. surgery involving the pelvis, predominately obstetrical and gynecological), plastic (e.g. surgery involving the restoration, reconstruction, correction or improvement in the shape and appearance of body structures that are defective, damaged or misshapened by injury, disease, or growth and development) or rectal (e.g. surgery of the rectum), urological (e.g. surgery related to the genitourinary system, predominately in males), vascular (e.g. surgery of the blood vessels), and surgery related to otolaryngology (e.g. surgery of the ears, nose, throat or related structures). The surgery can be conservative (e.g. surgery to preserve or remove with minimal risk, diseased or injured organs, tissues, or extremities) or radical (e.g. surgery designed to extirpate all areas of locally extensive disease and adjacent zones of lymphatic drainage).

As used herein, the term “cardiac surgery” has its general meaning in the art and is meant to encompass any surgery involving the heart, including but not limited to septal defect repair, inflow/outflow tract or valve procedure, heart valve repair or replacement, surgery to place ventricular assist devices or total artificial hearts, aneurysm repair, arrhythmia treatment, and the like.

As used herein, the term “cardiopulmonary by-pass” or “CPB” has its general meaning in the art and refers to a form of extracorporeal circulation whose function is circulatory and respiratory support along with temperature management to facilitate cardia surgery. CPB circuit includes pumps, cannulae, tubing, reservoir, oxygenator, heat exchanger and arterial line filter Modern CPB machines have systems for monitoring pressures, temperature, oxygen saturation, haemoglobin, blood gases, electrolytes as well as safety features such as bubble detectors, oxygen sensor and reservoir low-level detection alarm.

As used herein, the term “postoperative outcome” refers to the likelihood that the patient has at least one postoperative complication after an operation, in particular CPB. Typically, postoperative complications are related to tissue any damage or organ failure due to the release of pro-inflammatory cytokines and oxidative stress by circulating leucocytes in response to both ischemia and exposure to extra-corporeal artificial surface. In particular, postoperative outcome thus includes but is not limited to organ failure, acute atrial fibrillation and acute kidney injury.

As used herein, the term “organ failure” has its general meaning in the art and refers to a condition where an organ does not perform its expected function. Organ failure relates to organ dysfunction to such a degree that normal homeostasis cannot be maintained without external clinical intervention. Examples of organ failure include without limitation renal failure, liver failure, heart failure, and respiratory failure. Typically, organ failure is assessed by the Sequential Organ Failure Assessment (SOFA) score that is a simple and objective score that allows for calculation of both the number and the severity of organ dysfunction in six organ systems (respiratory, coagulatory, liver, cardiovascular, renal, and neurologic).

As used herein, the term “acute kidney injury” or “AKI” has its general meaning in the art and refers to loss of kidney function that develops within 6 days, e.g. following cardiac surgery. Kidney function may be assessed by glomerular filtration rate (GFR), i.e. the flow rate of filtered fluid through the kidney (for example, by the RIFLE class system, a GFR decrease >25% from baseline classifies risk, while injury is defined by a GFR >50% from baseline, as described in Nature Reviews Nephrology 7, 201-208; April 2011), or by creatinine clearance rate (C& or CrCl), i.e. the volume of blood plasma that is cleared of creatinine per unit time. Thus, loss of kidney function may be determined by an increase in blood levels of creatinine, e.g. a 50% or greater increase in creatinine concentrations. Typically, the AKI is assessed by the kidney disease improving global outcomes (KDIGO) score.

As used herein, the term “atrial fibrillation” has its general meaning in the art and refers to an arrhythmia in which the atrium is irregularly excited at a frequency of 450 to 600 times per minute, and that excitation wave is randomly transmitted to atrioventricular node, thus making the ventricular excitation irregular.

The method of the present invention is also suitable for predicting the length of stay. As used herein, the term “length of stay” means the amount of time the patient when the patient stays at hospital (e.g. in the intensive care unit). More particularly, the method of the present invention is also particularly suitable for predicting death of the patient.

In some embodiments, the present invention relates to a method of predicting the postoperative outcome of a patient after an acute circulatory failure induced by cardiopulmonary by-pass (CPB) comprising determining the level of redox potential in a sample obtained from said patient, wherein a downregulation of said redox potential as compared to a predetermined reference value indicates that the patient has a poor postoperative outcome.

In some embodiments, the present invention relates to a method of predicting the postoperative outcome of a patient after an acute circulatory failure induced by cardiopulmonary by-pass (CPB) comprising determining the level of redox potential in a sample obtained from said patient, wherein an upregulation of said redox potential as compared to a predetermined reference value indicates that the patient has a poor postoperative outcome.

In some embodiments, the sample is a whole blood sample. In some embodiments, the sample is an arterial whole blood sample. In some embodiments, the present invention relates to a method of predicting the postoperative outcome of a patient under cardiopulmonary by-pass (CPB) comprising determining the level of redox potential in a whole blood sample obtained from said patient, wherein an upregulation of said redox potential as compared to a predetermined reference value indicates that the patient has a poor postoperative outcome.

In some embodiments, said redox potential is measured at least 10 minutes after the initiation of cardiopulmonary by-pass (CPB). In some embodiments, said redox potential is measured at between 9 and 11 minutes after the initiation of cardiopulmonary by-pass (CPB). In some embodiments, said redox potential is measured 10 minutes after the initiation of cardiopulmonary by-pass (CPB).

In some embodiments, the level of redox potential is at least 20% higher than said predetermined reference value. In some embodiments, the level of redox potential is comprised between 83.5 and 114.3 mV. In some embodiments, the level of redox potential is comprised between 53.2 and 78.2 mV. In some embodiments, the level of redox potential is comprised between 138.1 and 149.5 mV. In some embodiments, a level of redox potential comprised between 138.1 and 149.5 mV indicates that said patient has a poor postoperative outcome.

In some embodiments, the predetermined reference value is a redox potential value obtained from a sample of said patient before a surgery. In some embodiments, the redox potential value obtained from a sample of said patient before surgery is between 78.1 and 117.5 mV. In some embodiments, the redox potential value obtained from a sample of said patient before surgery is between 109.7 and 147.8 mV. In some embodiments, the redox potential value obtained from a sample of said patient before surgery is between 73.6 and 92.3 mV.

In some embodiments, the present invention relates to a method of predicting the postoperative outcome of a patient after an acute circulatory failure induced by cardiopulmonary by-pass (CPB) comprising determining the level of redox potential in a whole blood sample obtained from said patient at least 10 minutes after the initiation of cardiopulmonary by-pass, wherein an upregulation of said redox potential of at least 20% as compared to a redox potential value obtained from a sample of said patient before said cardiopulmonary by-pass indicates that the patient has a poor postoperative outcome.

In some embodiments, the present invention relates to a method of predicting the postoperative outcome of a patient after an acute circulatory failure induced by cardiopulmonary by-pass (CPB) comprising determining the level of redox potential in a whole blood sample obtained from said patient at least 10 minutes after the initiation of cardiopulmonary by-pass, wherein a level of redox potential between 138.1 mV and 149.5 mV indicates that the patient has a poor postoperative outcome.

The invention will be further illustrated by the following figures and examples. However, these examples and figures should not be interpreted in any way as limiting the scope of the present invention.

### FIGURES

FIG. 1. Arterial oxidation-reduction potential during CPB.

FIG. 2. Macro- and micro-hemodynamic data during CPB. (A) Arterial lactate/pyruvate ratio. (B) NIRS value (muscle). (C) Systolic Blood Pressure (SBP).

FIG. 3. Variation of acetoacetate/p-hydroxybutyrate ratio during CPB.

FIG. 4. Identification of three categories of patients under CPB depending on the variation of the oxidation-reduction potential. Redox potential expressed as a percentage of the pre-CPB value. (A) Return to Basal Eg Value (RBV). (B) Eg at t=10 min 20% higher than the basal value (SUP). (C) Eg lower by 20% (INF).

FIG. 5. (A) Comparison between oxidation-reduction potential and (B) lactate/pyruvate ratio (t=10 min).

### EXAMPLE

### Material and Methods

### Description of the Study

A prospective, observational, monocentric study was conducted in the cardiac surgery department of the Centre Hospitalier Universitaire (CHU) de Toulouse. All the data were collected over a period extending from Jan. 28, 2022 to May 19, 2022. This project was part of the ODCOTA protocol focusing on postoperative cognitive dysfunction following cardiac surgery with extracorporeal circulation and during which perioperative samples are taken (NCT04907565; RC31/21/0014). The consent of the patients is therefore obtained before their inclusion in the study.

### Population

The patients included in the study presented the following criteria:
Cardiac surgery scheduled under general anesthesia requiring the establishment of extracorporeal circulation
Patients over 60 years old
Preoperative MMSE (Mini-Mental State Examination) greater than or equal to 20/30
Patients able to provide their informed consent.

The exclusion criteria were:
Urgent surgery or septic context
Patients with known dementia or an MMSE score below 20/30
Minor patients, under protection, pregnancy or breast-feeding.

### Circulatory Failure Model

Our model of circulatory insufficiency is associated to the initiation of extracorporeal circulation. During the extracorporeal circulation, the patient's blood is suddenly diverted into the extracorporeal circulation. This transition period is associated with arterial hypotension (10,11). The patient's spontaneous cardiac output drops and the blood flow is then ensured by the CPB. The initiation of the CPB provides a programmed circulatory insufficiency.

### Judging Criteria

Our primary endpoint was the measurement of Eg in arterial whole blood.

The secondary judgment criteria were:
The demonstration of acute circulatory insufficiency at the initiation of the CPB: a lactate/pyruvate ratio greater than 20, a reduction in the values of muscular NIRS, as well as a reduction in blood pressure as compared to the value before CPB
Measurement of the concentration of ketone bodies (acetoacetate and β-hydroxybutyrate)
The association between organ dysfunction and variability of the oxidation-reduction potential: the patients were classified according to their ability to recover or not their basal oxidation-reduction potential (pre-CPB) value at 10 min after the initiation of CPB, taking into account a variability around the 20% measurement.

### Research Procedure

The preparation for the intervention followed the national recommendations (12). The patient benefited from the usual care: namely the implementation of the monitoring elements (scope electrodes, O₂ saturation). After pre-oxygenation with pure oxygen, the patient was anesthetized according to the protocol in force in the department including: propofol, sufentanil, cisatracurium, ketamine, sulphate magnesium and lidocaine, as well as common analgesics such as paracetamol, ketoprofen, and acupan. The patient was then mechanistically ventilated. The inspired fraction of O₂ was adapted to reach 95% saturation. A central jugular line was placed as well as a left radial arterial catheter. All of the surgeries were performed by stemotomy. After heparinization of the patient, CPB was started. The blood circulating in the CPB system was warmed (37° C.), oxygenated (PO₂ 140 mmHg), and returned with increasing flow into the aorta through the arterial cannula. When the extracorporeal circulation reached the target rate (2.4 ml/min/m2 according to DUBOIS formula), cardiac activity was gradually stopped and heart surgery began. Once the surgical procedure was completed, the coronary arteries were reperfused with oxygenated blood, the heart was put back on charge and the CPB gradually weaned off. Once the patient's chest was closed, the patient was conducted to post-surgical intensive care. Next morning (D+1), the measurements were repeated. At the end of hospitalization, hospitalization data were collected, with particular attention to any complications during treatment in the theater and intensive care.

During the procedure, several blood samples were taken from the radial arterial catheter. On these samples the following measurements were taken:
Measurement of blood pH, O₂, and CO₂ partial pressures, arterial oxygen saturation
Measurement of global arterial Eg. The Eg measurement is performed using an electrode (13). The measuring electrode was immediately immersed in the patient's whole arterial blood without pre-treatment. Data were collected for 75 seconds using the NOVA® 2.0 application. The potential value was obtained after stabilization (approximately 30 seconds)
Determination of the lactate and pyruvate concentration on perchloric acid tubes stored at +4° C. immediately after sampling.

The samples were taken at the following sequences:
The 1^{st} sample was taken under general anesthesia before the start of the CPB (“Pre-CPB”)
The 2^{nd} sample was taken as soon as the CPB target rate was reached (“0 min”)
Samples were taken every 10 minutes during the first 30 minutes of the CPB
A sample was taken at the end of the CPB
A sample was taken on postoperative D+1.

Finally, a NIRS electrode was added on the left deltoid or the left quadriceps in order to continuously measure the level of muscle tissue oxygenation.

### Statistical Analysis

Binary variables were expressed as a percentage. Continuous variables were expressed as mean and standard error. The analyzes were carried out using GraphPAD Prism® 8.0 software. The univariate statistical tests were carried out by the Mann-Whitney method for continuous variables and by a Filer test for binary variables. A value of p<0.05 was considered statistically significant.

### Results

The initiation of extracorporeal circulation (“0 min”) was used as a model of circulatory insufficiency.

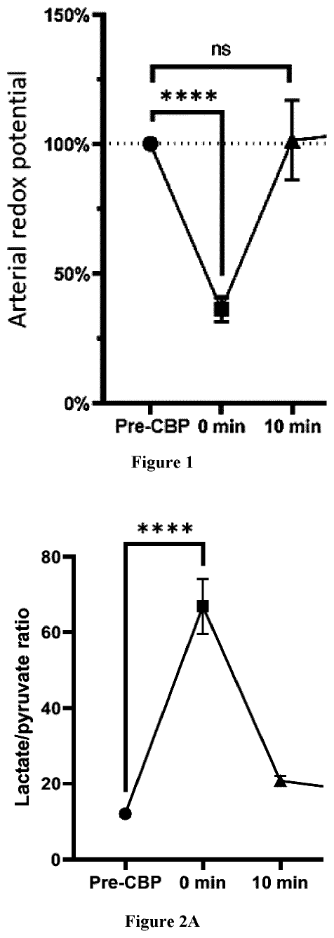

FIG. 1 demonstrates a significant reduction in the redox potential when t=0 min, as compared to the potential under general anesthesia before CPB (100%). The median of this decrease is 36.6% (+/−1.09; p<). We noted in most of cases (41.2%) a rapid normalization of this oxidation-reduction potential (return to 100%+/−20%) at t=10 min.

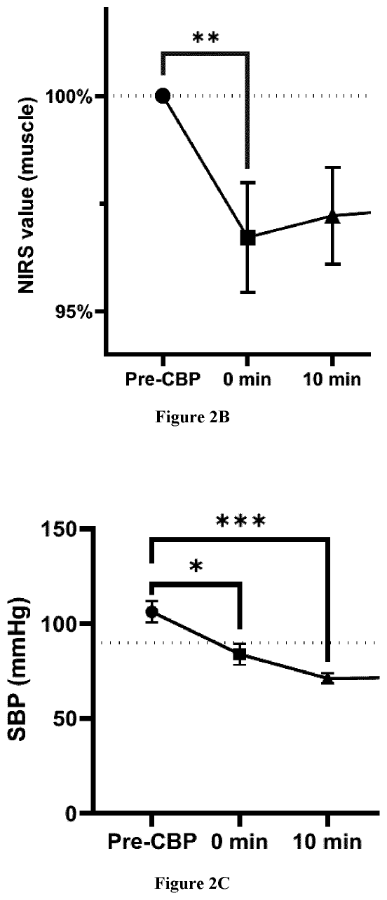

FIG. 2 demonstrates that the initiation (t=0 min) of CPB induced an increasement both in hyperlactatemia (540%+/−220%; p=<0.0001) and in lactate/pyruvate ratio (66.8+/−31.3 vs 12.1+/−2.8; p=0.0001) (FIG. 2A) at baseline. CPB also seems to affect skeletal muscle tissue perfusion with a significant variation in tissue saturation (72%+/−3.8 vs 69.7%+/−6.12 before CPB; p=0.0073) (FIG. 2B). The initiation of CPB also seemed to lead to a significant decrease in SBP at t=0 and t=10 min (106.3 mmHg+/−26.1 vs 83.9+/−25.9 at t=0 min and 71.2+/−11.1 at t=10 min; p=0.014 and p=0.0004 respectively) (FIG. 2C), with no impact on MAP (75.2 mmHg+/−17.2 versus 72.5+/−19.0; p=0, 0004).

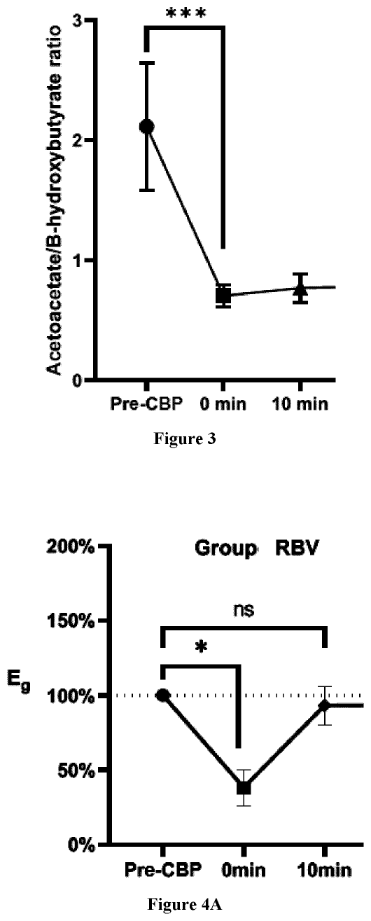

FIG. 3 demonstrates that acetoacetate/p-hydroxybutyrate ratio decreases significantly during CPB initiation (2.11+/−2.25 during pre-CPB versus 0.70+/−0.40 at t=0 min; p=0.0002). The ratio remains relatively low thereafter. We therefore highlight an alteration of the hepatic mitochondrial function probably related to the circulatory insufficiency related to the establishment of the CPB.

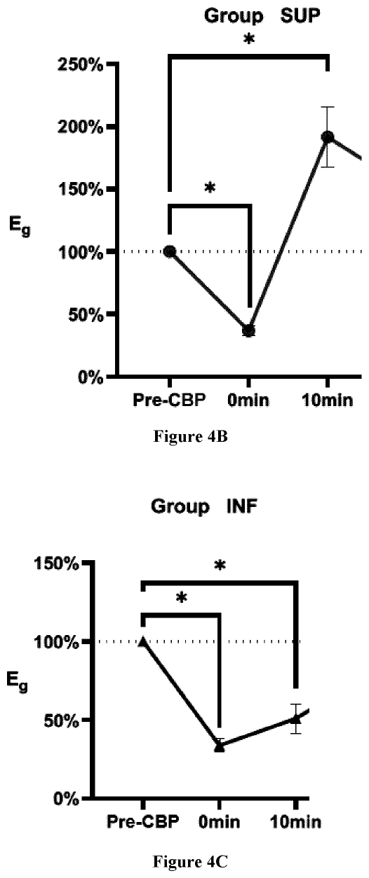

In order to establish whether the arterial redox potential variability and the postoperative organ failure are linked, we classified the patients according to the difference between their Eg value at t=0 min (pre-CPB) and t=10 min. FIG. 4 shows that we have distinguished three groups (Table 1):
RBV group: Eg returned to the pre-CPB basal value at t=l0 min (+/−20%) (FIG. 4A)
SUP group: Eg with a value that exceeds the basal value by more than 20% at t=10 min (SUP) (FIG. 4B)
INF group: Eg with a value lower than 20% as compared to the basal value at t=l0 min (INF) (FIG. 4C).

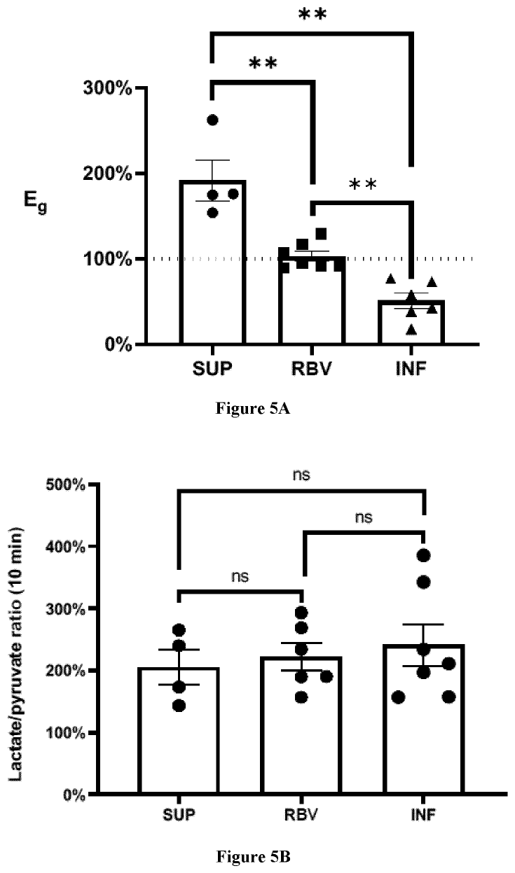

FIG. 5 demonstrates that this classification is specific to the variability of arterial redox potential. Indeed, the comparison between the values of the three groups at t=10 min was statistically significant (FIG. 5A). This discrimination was not found with the lactate/pyruvate ratio values (FIG. 5B). IGS II was also lower for patients in the INF group (43.5+/−12.8 versus 25+/−6.06; p=0.036) and atrial fibrillation was over-represented in patients belonging to the SUP group (75% versus 16.7%; p=0.033).

### CONCLUSION

We made a prospective study with 17 patients eligible for cardiac surgery with cardiopulmonary bypass. We showed a frank reduction of the whole blood redox potential during circulatory failure at the initiation of CPB (−36%+/−1.09; p<0.0001). We also showed that the whole blood potential might evolve differently at 10 minutes after CPB initiation. Some patients (i.e. SUP group) seems to present an unfavorable post-operative outcome (SPASII 43.5+/−12.8 vs 25+/−6.06; p=0.036). In conclusion, whole blood potential is a new feature for circulatory failure monitoring during CPB.

### TABLES

TABLE 1
E_{g} variations between the three identified groups

| | | | E_{g} value | Mean percent |
|---|---|---|---|---|
| | | E_{g} basal | (t = 10 min | change from |
| Groups | n | value (mean) | (mean) | baseline |
| RBV | 7 | 99.34 (78.1- | 99.51 (83.5- | +2.87 (+/−15.1) |
| | | 117.5) | 114.3) | |
| INF | 6 | 128.10 (109.7- | 64.80 (53.2- | −49.1 (+/−22.8) |
| | | 147.8) | 78.2) | |
| SUP | 4 | 80.41 (73.6- | 147.3 (138.1- | +91.6 (+/−48) |
| | | 92.3) | 149.5) | |

### REFERENCES

Throughout this application, various references describe the state of the art to which this invention pertains. The disclosures of these references are hereby incorporated by reference into the present disclosure.
1. Vincent J L, De Backer D. Circulatory Shock. Finfer S R, Vincent J L, éditeurs. N Engl J Med. 31 Oct. 2013; 369(18):1726-34.
2. Fiaccadori E, Vezzani A, Coffrini E, Guariglia A, Ronda N, Tortorella G, et al. Cell metabolism in patients undergoing major valvular heart surgery: Relationship with intra and postoperative hemodynamics, oxygen transport, and oxygen utilization patterns. Crit Care Med. déc 1989; 17(12):1286-92.
3. Cecconi M, De Backer D, Antonelli M, Beale R, Bakker J, Hofer C, et al. Consensus on circulatory shock and hemodynamic monitoring. Task force of the European Society of Intensive Care Medicine. Intensive Care Med. déc 2014; 40(12):1795-815.
4. Meng L. Heterogeneous impact of hypotension on organ perfusion and outcomes: a narrative review. Br J Anaesth. déc 2021; 127(6):845-61.
5. Rivers E, Nguyen B, Havstad S, Ressler J, Muzzin A, Knoblich B, et al. Early Goal-Directed Therapy in the Treatment of Severe Sepsis and Septic Shock. N Engl J Med. 8 Nov. 2001; 345(19):1368-77.
6. Tongers J, Sieweke J T, Kühn C, Napp L C, Flierl U, Röntgen P, et al. Early Escalation of Mechanical Circulatory Support Stabilizes and Potentially Rescues Patients in Refractory Cardiogenic Shock. Circ Heart Fail. mars 2020; 13(3):e005853.
7. Lassus J, Tarvasmäki T, Tolppanen H. Biomarkers in cardiogenic shock. Adv Clin Chem. 2022; 109:31-73. doi: 10.1016/bs.acc.2022.03.002. Epub 2022 Apr. 25. PMID: 35953128.
8. Weinberger J, Klompas M, Rhee C. What Is the Utility of Measuring Lactate Levels in Patients with Sepsis and Septic Shock?Semin Respir Crit Care Med. October 2021; 42(05):650-61.
9. Rimachi R, De Carvahlo F B, Orellano-Jimenez C, Cotton F, Vincent J L, De Backer D. Lactate/Pyruvate Ratio as a Marker of Tissue Hypoxia in Circulatory and Septic Shock. Anaesth Intensive Care. mai 2012; 40(3):427-32.
10. Murphy G S, Hessel E A, Groom R C. Optimal Perfusion During Cardiopulmonary Bypass: An Evidence-Based Approach. Anesth Analg. mai 2009; 108(5):1394-417.
11. Barry A E, Chaney M A, London M J. Anesthetic Management During Cardiopulmonary Bypass: A Systematic Review. Anesth Analg. avr 2015; 120(4):749-69.
12. Carillion A, Rozencwajg S, Abou Arab O, Fischer M O. Enhanced recovery after cardiac surgery under CPB or off pump. Anaesth Crit Care Pain Med. août 2022; 41(4):101106.
13. Harris A R, Carter P, Cowan R, Wallace G G. Impact of Protein Fouling on the Charge Injection Capacity, Impedance, and Effective Electrode Area of Platinum Electrodes for Bionic Devices. Chem Electro Chem. 12 mars 2021; 8(6):1078-90.

1. A method of diagnosing and treating acute circulatory failure in a patient in need thereof comprising
measuring a level of redox potential in a sample obtained from said patient,
determining that the redox potential is downregulated compared to a reference value, and
treating the subject determined to have a downregulated redox potential for acute circulatory failure.

2. The method according to claim 1 wherein the sample is a whole blood sample.

3. The method according to claim 1 wherein a downregulation of said redox potential as compared to a predetermined reference value indicates that the patient suffers from acute circulatory failure.

4. A method of predicting the outcome of a patient after an acute circulatory failure and treating the patient comprising
measuring a level of redox potential in a sample obtained from said patient,
determining that the level of the redox potential is upregulated compared to a predetermined reference value, and
treating the patient identified as having an upregulated redox potential value with a treatment suitable for a patient with a poor outcome.

5. The method according to claim 4 wherein the acute circulatory failure is induced by cardiopulmonary by-pass surgery.

6. The method according to claim 4 wherein the sample is a whole blood sample.

7. The method according to claim 4 wherein an upregulation of said redox potential as compared to a predetermined reference value indicates that the patient has a poor outcome.

8. The method according to claim 5 wherein said redox potential is measured at least 10 minutes after the initiation of cardiopulmonary by-pass (CPB).

9. The method according to claim 7 wherein the level of redox potential is at least 20% higher than said predetermined reference value.

10. The method according to claim 5 wherein the predetermined reference value is a redox potential value obtained from a sample from said patient before a surgery.

11. The method according to claim 5 wherein a level of redox potential comprised between 138.1 and 149.5 mV indicates that said patient has a poor postoperative outcome.

12. The method according to claim 5 comprising determining the level of redox potential in a whole blood sample obtained from said patient at least 10 minutes after the initiation of cardiopulmonary by-pass, wherein an upregulation of said redox potential of at least 20% as compared to a redox potential value obtained from a sample of said patient before said cardiopulmonary by-pass indicates that the patient has a poor postoperative outcome.

13. The method according to claim 5 comprising determining the level of redox potential in a whole blood sample obtained from said patient at least 10 minutes after the initiation of cardiopulmonary by-pass, wherein a level of redox potential between 138.1 mV and 149.5 mV indicates that the patient has a poor postoperative outcome.
